(19) United States
(12) Patent Application Publication
(10) Pub. No.: US 2011/0318773 A1
(43) Pub. Date: Dec. 29, 2011

(54) METHOD TO USE EKTACYTOMETRY TO IMPROVE STORED RED BLOOD CELL OR WHOLE BLOOD PRODUCT UTILIZATION

(76) Inventors: Michael Tarasev, Pinckney, MI (US); Kenneth Alfano, Canton, MI (US)

(21) Appl. No.: 12/822,484

(22) Filed: Jun. 24, 2010

(51) Int. Cl. C12Q 1/02

(52) U.S. Cl. 435/29

(57) ABSTRACT

A method for assessing the quality degradation of an RBC or whole blood unit, the method comprising: 1) using an ektacytometer to generate erythrocyte deformability data for the RBC or whole blood unit; 2) analyzing the erythrocyte deformability data to assess the quality degradation of the RBC or whole blood unit; 3) providing the erythrocyte deformability data to medical personnel; 4) correlating the erythrocyte deformability data with a clinical outcome of a patient that has used the RBC or whole blood unit; and 5) incorporating the erythrocyte deformability data into subsequent assessments of quality degradation of RBC and whole blood units. This method would provide clinicians with actual data on RBC quality when making decisions about which and how many units to use for transfusion of a given patient. Moreover, deploying the approach throughout the supply chain will improve distribution, planning, and inventory control decisions.

### FIELD OF THE INVENTION

The present disclosure is in the technical field of blood banking and transfusion services. More particularly, the present disclosure is in the technical field of quality control and management of stored Red Blood Cell (RBC) or whole blood units.

### BACKGROUND OF THE INVENTION

Blood transfusions are used for a wide variety of patients under many circumstances. Most blood transfusions are, in fact, transfusions of red blood cells. Red blood cells are stored in Red Blood Cell (RBC) units. The blood banking industry, transfusion industry, and hospitals monitor RBC units. The current maximum age for transfusable RBC units is 42 days. RBC units are typically administered on a first-in first-out (FIFO) basis.

### BRIEF SUMMARY OF THE INVENTION

The present disclosure describes a method for assessing the quality degradation of individual stored Red Blood Cell (RBC) or whole blood units, thereby yielding information to improve decisions regarding their respective allocation, patient suitability, and use.

The method for assessing the quality degradation of an RBC or whole blood unit comprises: using an ektacytometer to generate erythrocyte deformability data for the RBC or whole blood unit; analyzing the erythrocyte deformability data to assess the quality degradation of the RBC or whole blood unit; providing the erythrocyte deformability data to medical personnel; correlating the erythrocyte deformability data with a clinical outcome of a patient that has used the RBC or whole blood unit; and incorporating the erythrocyte deformability data into subsequent assessments of quality degradation of RBC and whole blood units.

The first step in the method is using an ektacytometer to generate erythrocyte deformability data for the RBC or whole blood unit. An ektacytometer uses laser-assisted diffraction to measure the deformability of a cell. Ektacytometry is a long-established technique for measuring the ability of a cell to deform under relatively mild stresses; such “deformability” is in contrast to either osmotic or mechanical “fragility,” which use higher stresses to assess cells' propensity for lysis (breakage). In an alternate embodiment, a different apparatus or method can be substituted for the ektacytometer.

The next step in the method is analyzing the erythrocyte deformability data to assess the quality degradation of the RBC or whole blood unit. In this step, the deformability data is compared to previous deformability data which has already been correlated to a quality degradation value. Hence, the deformability data for a given sample can be given a quality degradation value based on prior correlations.

The next step in the method is providing the erythrocyte deformability data to medical personnel. Medical personnel can include doctors, nurses, lab technicians, and the like.

The next step in the method is correlating the erythrocyte deformability data with a clinical outcome of a patient that has used the RBC or whole blood unit. Medical personnel can assist in the correlation, since clinical outcomes can be both subjective and objective.

The final step in the method is incorporating the erythrocyte deformability data into subsequent assessments of quality degradation of RBC and whole blood units. This recursive approach allows for continual improvement of the metrics used to assess the quality degradation of an RBC or whole blood unit.

In an alternate embodiment, the deformability data comprises aggregate membrane deformability as a function of time. Hence, the deformability data can be used to estimate the effect that storage will have upon a given RBC or whole blood unit. Also, the deformability data can be used to assess storage methods and lead to storage improvements.

In an alternate embodiment, the deformability data comprises the rate of change of membrane deformability as a function of time. This enables an estimate of whether the quality degradation of RBC and whole blood units accelerates or decelerates with time.

In an alternate embodiment, the method further comprises using deformability data to generate a value representing RBC or whole blood unit suitability for transfusion or degree of prospective efficacy.

In an alternate embodiment, the method further comprises assessing the quality degradation of an RBC or whole blood unit in some combination with RBC fragility, morphologic RBC parameters, or metabolic RBC parameters to evaluate the RBC or whole blood unit.

In an alternate embodiment, the method further comprises using the quality degradation of an RBC or whole blood unit to aid management of blood supply or inventory.

In an alternate embodiment, the method further comprises using the quality degradation of an RBC or whole blood unit to evaluate blood, storage and handling methods.

In an alternate embodiment, the method further comprises using the quality degradation of an RBC or whole blood unit to aid RBC or whole blood usage in triage.

In an alternate embodiment, the method further comprises using the quality degradation of an RBC or whole blood unit to make indication-specific assessments of RBC unit suitability for specific patient groups, based on particularized efficacy correlations found for such patient groups.

Upon validation, this method is amenable to clinical implementation yielding information indicative of any given unit's relative quality and thus prospective efficacy. This would provide clinicians with actual data on RBC quality to consider when making decisions about which and how many units to use for transfusion of a given patient. Moreover, deploying this approach throughout the supply chain will improve distribution, planning, and inventory control decisions via established Operations Research (OR) principles. An element of this approach is the accumulation of copious output and other associated data—and the mathematical/computational analyses thereof—to optimize and refine algorithms which interpret subsequent output to characterize each unit of blood as meaningfully as possible. Although ektacytometry and related methods have existed for many years, only diagnostic applications have so far been pursued in the art. Relevant RBC membrane properties—broadly referred to as “deformability,” despite considerable qualitative differences among testing techniques—show plausible potential to represent storage-induced degradation (“storage lesion”). Despite suspicion in the art beginning three decades ago that ektacytometry might potentially be useful for predicting transfusion efficacy, this possibility has remained unexplored (and the need unresolved), due in part to broad skepticism, as well as lack of any a methodical approach to clinically-actionable implementation.

Although deformability is likely to be shown a somewhat less relevant and beneficial property than fragility for characterizing stored blood or predicting transfusion success, there are nonetheless circumstances in which it could be a welcome alternative and/or supplement.

The computational steps analyze the deformability data for a given sample, considering a progressively accumulating body of data gathered correlating such data to clinical outcomes. The output information reflects the relative degradation (and thus the prospective transfusion efficacy) of any given unit in an inventory, which can be usefully considered by clinicians, blood bankers, or others responsible for RBC allocation or use.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

The following detailed description of the invention is merely exemplary in nature and is not intended to limit the invention or the application and uses of the invention. Furthermore, there is no intention to be bound by any theory presented in the preceding background of the invention or the following detailed description of the invention.

### BRIEF DESCRIPTION OF THE DRAWINGS

FIG. 1 shows a method for assessing the quality degradation of an RBC or whole blood unit

### DETAILED DESCRIPTION OF THE INVENTION

During storage, RBC quality degrades due to a number of morphological and biochemical changes in the RBC, including ATP depletion and loss of endogenous RBC antioxidants, leading to damage of RBC cytoskeletal proteins and the membrane in general, resulting in decreased RBC viability in vivo upon transfusion. Such RBC degradation is reflected in the compromised deformability and increased fragility of the RBC membrane, which are both inversely related to post-transfusion RBC survival and tissue-oxygenation efficacy.

Research indicates that for certain patient groups (e.g. critical care) many RBC units can become dangerously ineffective well before their 42-day maximum age. The exact age when this occurs varies among RBC units, and there is currently no means of testing specific RBC units for such quality loss. The degradation of any given RBC unit varies with several contributing factors in addition to time—including donor-to-donor variability, storage conditions, and transportation conditions, among others—making the current 42-day uniform age-standard an inadequate proxy.

The extent of degradation is critical for certain patient groups, and if known, physicians could make better-informed treatment judgments. For example, critical-care patients who require immediate restoration of tissue oxygenation are the most clearly harmed by transfusion inefficacy, and thus may warrant priority for the most viable units. However, there is so far no way to discriminate for viability among non-outdated units. Conversely, slowly-degrading units could be acceptable for some patients even beyond 42 days, but without individualized testing, there is no way to identify such units.

With no reliable predictor of transfusion efficacy for any given RBC unit, physicians sometimes withhold transfusions from those patients whom they believe can recover without the transfusion (“restrictive” approach). But such a practice can potentially delay patient recovery, increase hospital stays, increase the need for additional procedures, and increase a patient's risk. However, the physician withholding the transfusion may feel that the potential for complications from using blood with unknowable viability outweighs those other issues. In other cases, physicians are sometimes forced to use more units than would otherwise be necessary, in order to minimize the chance of failing to provide enough viable RBC quickly enough to restore tissue oxygenation immediately (“liberal” protocol). This not only consumes additional scarce units that might have been suitable for other (less-critical) patients, but also subjects the patient to risks of various complications. Some risks such as type-match errors are simply proportional to the number of units being transfused; others—like volume overload—are specifically associated with receiving excessive blood. In fact, some hospitals currently attempt to accommodate case-by-case requests from trauma surgeons for “fresher” blood, but scarcity renders this rare. And aside from issues of patient safety or wasted units, considerable costs are associated with all current practices.

In addition, present methods of blood banking supply chain and inventory management are likewise unable to take into account the relative degradation levels or rates of particular RBC units, but must instead default to “first-in-first-out” (FIFO). Lacking any means of measuring and tracking actual quality, the poor proxy provided by mere time-in-storage leads to suboptimal routing, distribution, and allocation.

Red blood cells (also known as erythrocytes; when capitalized, “Red Blood Cells” or RBC specifically refers to the stored biologic) are highly-specialized cells responsible for delivery of oxygen to, and removal of carbon dioxide from, metabolically-active cells via the capillary network. They are shaped as biconcave discs and average about 8-10 microns in diameter. The membrane is very flexible so as to allow the cell to travel in capillaries with diameters of only 4-5 microns. At the same time, the membrane must be strong enough to withstand significant ongoing flow-induced stresses while avoiding tears or fragmentation. An erythrocyte with normal membrane stability and plasticity is able to circulate effectively and without damage, whereas a degraded cell is likelier to suffer hemolysis or plug capillaries in vivo.

A variety of anticoagulant and preservative (A-P) solutions have been developed to enable long-term storage. RBC units in liquid state are stored at 1-6° C., with a current maximum FDA-permitted shelf life of 42 days. A significant proportion of patients receive blood products substantially affected by storage; the average age of RBC transfused in the US is 21 days (higher for rare blood types).

Studies diverge on the question of how storage time impacts transfusion efficacy. Several preclinical trials link higher storage times to lower tissue oxygenation.

Increased storage time has also been implicated in increased incidents of mortality, pneumonia, post-injury multiple organ failure, hemorrheological disorders, serious infections, TRALI, and adverse microcirculatory hemodynamics. Many reviews analyzing the effect of RBC storage on transfusion efficacy questions the net benefit of using stored RBC in the critically-ill.

On the other hand, a number of studies notably did not detect an adverse effect of RBC storage time on transfusion efficacy. This has led to an increasing and unresolved controversy about whether “older” blood is less beneficial than “newer” blood. Several hypotheses have been proposed to explain this inconsistency—including insufficient ranges among storage times, the use of mixed/multiple RBC units in any given procedure, the potential effect of white blood cell burden, variable patient physiological conditions, and the idea that storage time alone is an incomplete indicator of quality.

Prolonged storage of RBC results in an array of morphological and biochemical changes, collectively referred as “storage lesion,” associated with depletion of ATP and 2,3-diphosphoglycerate (2,3-DPG) levels and increased oxidative stress. Also reported is a decrease in RBC deformability (although studies vary widely on when this occurs), a process mediated by storage-induced oxidative injuries and changes in metabolic state. Thus, the condition of the membrane offers the capacity to serve as an aggregate indicator of overall cell viability and prospective performance.

The magnitudes of the observed RBC membrane changes appear to depend on a variety of factors besides time, including A-P solution used, the presence of modifying additives, bag material, etc. This issue is further complicated by results indicating that properties of RBC solutions toward the end of their shelf life (including in-vitro hemolysis) were largely dependent upon conditions of production, storage, and/or transport by the manufacturer. This variability may be also related to the presence of other formed elements in the solutions. Variability among RBC properties from different donors adds an additional unknown parameter to degradation levels and/or rates. RBC deformability loss has been extensively documented by a variety of experimental techniques including micropipette techniques, micropore filtration, optical tweezers, and laser-assisted diffractometry (a.k.a. ektacytometry).

It should be noted that although in most cases the results are presented and discussed in terms of cell “deformability,” the underlying properties measured by these various tests are often qualitatively variant. Also, some techniques measure properties averaged throughout a given sample, while others derive results from single-cell measurement. Low-stress, single-cell deformability tests have long been pursued in clinical diagnostic applications, and have been suspected to have potential in transfusion medicine as early as 1982* and 1983**, but such suggestions went unexplored for decades despite growing concerns about the quality of stored blood, because the field remained persistently skeptical that any in vitro RBC test could successfully be used to predict in vivo cell behavior. Hence, no methods have yet been formulated to exploit this potential to meet the longstanding need for a reliable “time-independent” measure of RBC quality in addition to storage time. While the controversy about stored blood degradation thus far has focused almost exclusively on time/age, the present invention introduces the use of in vitro properties which some data indicate offer the potential to supplement time/age as a quality metric.

Reduced deformability/plasticity in RBC has been shown to significantly affect both the post-transfusion survival time in the bloodstream, and cells' ability to traverse the capillary network. Stiffened RBC can significantly alter pulmonary hemodynamics, resulting in increased vascular resistance. Partially-hardened (albeit non-physiologically) cells were shown to disappear from circulation within 25 minutes after transfusion, compared with <2% of others; that study also indicated that reduced RBC deformability leads to cell entrapment in capillaries and microcirculatory blockage, impeding flow through certain regions of microcirculation.

A reduction in post-transfusion RBC viability is a well-established consequence of ex vivo storage. One accepted criterion of transfusion efficacy is >70% RBC survival 24 hours post-transfusion. FDA regulations actually now call for this level to be 75%. Currently, this is verified only at the development of the A-P solutions, but compliance is not ascertainable in clinical practice or on a per-unit basis. (Tracking post-transfusion RBC survival typically requires radio-labeling, which is only performed in limited research settings.) No clinical tests or processes are available to discriminate (by quality or prospective degree of performance) among available RBC units.

While there exist various purported means of measuring RBC membrane integrity (including deformability, plus mechanical and osmotic fragility), none have yet been correlated to transfusion outcomes or approached in a systematic manner for utilizing such correlations. There is no established “gold standard” or consensus metric for loss of RBC quality or viability. Known commercial efforts at this time directed at measuring red blood cell membrane integrity appear to be focused on deformability measurements on patient blood, for diagnosing pathologies known to be associated with such properties.

Notably, deformability (and thus ektacytometry) testing is expected to be inferior to (mechanical) fragility testing for evaluating RBC product quality, because of the qualitative difference between the former (measured via low-stress subjection, typically with single-cells) and the latter (measured mainly at higher stresses, for gauging aggregate cell propensity for lysis). Mechanical fragility is expected to be superior for two reasons: the nature of the stress is more representative of the physiological stresses erythrocytes experience in vivo, plus the nature of fragility testing enables multiple independent parameters to be varied concurrently for a richer pool of relevant data. Nevertheless, techniques and devices for deformability are so well established and readily-adoptable in the transfusion field, that even a suboptimal correlation or predictive power could indeed prove useful in certain cases. In particular, it could supplement or complement the higher-stress, lysis-inducing (fragility-based) methods—or provide an additional or alternative source of data for quantifying the quality of stored RBC. In fact, deformability testing could be used in conjunction with mechanical fragility testing, whereby the latter stresses some cells to the point of hemolysis and the former looks separately at those cells which did not lyse in said testing. Also, for certain select kinds of anemias, deformability alone may even show superior relevance.

As indicated, the value of developing any test for RBC degradation correlated to clinical utility is presently disputed; some in the blood banking industry currently resist the suggestion that 1) RBC age/degradation is a clinical concern (within the current 42-day limit), or that 2) measuring the degradation of individual RBC units could improve decisions about their use. While data do exist to support both contentions, neither has yet been established. Except for the small sampling of RBC being tested for auto-lysis near outdating (regulated with a 1% maximum in the US), there is presently no systematic assessment of blood product degradation at all in clinical practice. Many experts doubt that any in vitro test would be able to predict in vivo cell survival or behavior—even declaring that such endeavors are not worth pursuing.

The present disclosure describes a methodical approach for testing the degradation levels of stored blood prior to transfusion that is conducive to clinical adoption for generating clinically-relevant information. The present disclosure describes a procedure for quantifying the quality degradation of individual stored red blood cell (RBC) units in terms of cell deformability (i.e. ektacytometry), and processing this data to facilitate better-informed medical decisions regarding units' respective allocation, patient suitability, and use. This would provide clinicians with presently-unavailable data on RBC quality when making decisions about which and how many units to use for transfusion to a given patient. Moreover, deploying this testing across a hospital's inventory or even throughout the supply chain will improve distribution, planning, and inventory control decisions. An aspect of this systematic approach is the recursive accumulation of copious output and other associated data (e.g. clinical outcomes) and the computational analyses thereof to optimize the characterization of all subsequent units.

Currently, “FIFO” (first-in-first-out) is the most common method of inventory planning. Certain deviations from FIFO do exist (e.g. for neonatal patients) but are overwhelmingly based on time-in-storage as the criteria for anticipating RBC in vivo performance (and thus transfusion efficacy). However, regularly-performed testing of prospective RBC viability for all units in an inventory would enable a quality-based ranking to supplement (or perhaps eventually replace) time-based ordering and distribution of RBC units. For example, a unit with a higher degradation level and/or rate would get used faster to preempt excessive quality loss; such a proactive practice would minimize overall net degradation before use. Additionally, the resulting increased net viability across all RBC units could reduce the number of units necessary to achieve the same clinical effect of blood transfusion on any given patient, thereby reducing the overall amount of blood used. Potential also exists for using RBC viability to establish unit expiration times based on actual blood quality, as opposed to a pre-set uniform deadline, thus increasing possible storage time of at least some blood and reducing blood loss through outdating (although this would require a major regulatory change). In another embodiment of this application method, the rates of deformability loss are themselves viewed as a quality metric, instead of or in addition to deformability levels.

Also, upon validation, a triaging application could enable diversion of low viability units from vulnerable patients and unit selection according to patient efficacy needs to potentially reduce post-transfusion complications and improve overall transfusion efficacy and clinical outcomes. Aside from matching higher-efficacy units with patients who most need them, it could also avoid “wasting” other units on patients whom they may not benefit. For example, it is possible that transfusions which would be deleterious in patients with normal erythrocyte deformability may still be beneficial when performed in patients with markedly altered deformability—particularly for a small oxygen deficit.

The method for assessing the quality degradation of an RBC or whole blood unit comprises: 1) using an ektacytometer to generate erythrocyte deformability data for the RBC or whole blood unit; 2) analyzing the erythrocyte deformability data to assess the quality degradation of the RBC or whole blood unit; 3) providing the erythrocyte deformability data to medical personnel; 4) correlating the erythrocyte deformability data with a clinical outcome of a patient that has used the RBC or whole blood unit; and 5) incorporating the erythrocyte deformability data into subsequent assessments of quality degradation of RBC and whole blood units.

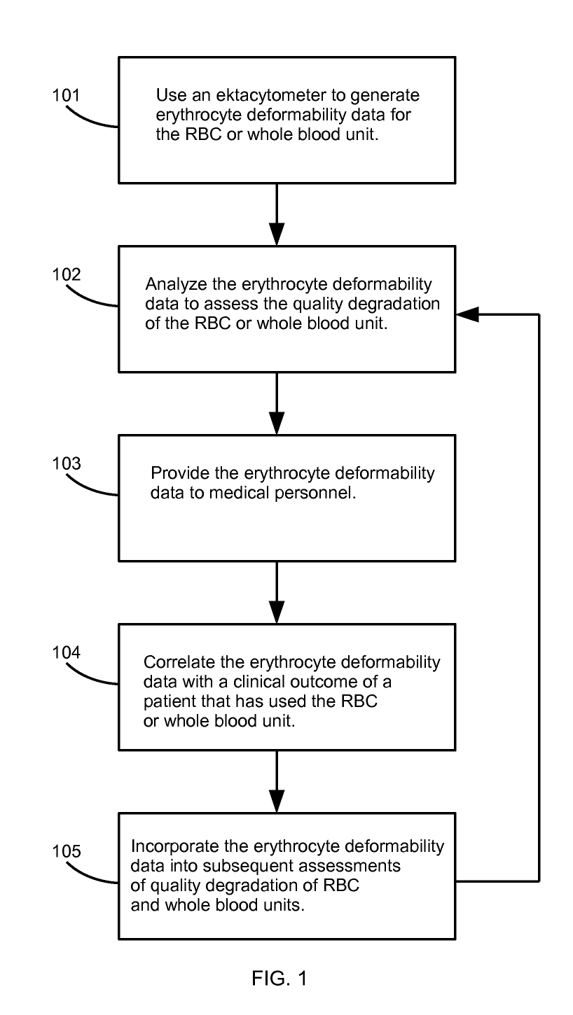

FIG. 1 shows method for assessing the quality degradation of an RBC or whole blood unit. Step 101 uses an ektacytometer to generate erythrocyte deformability data for the RBC or whole blood unit. Step 102 analyzes the erythrocyte deformability data to assess the quality degradation of the RBC or whole blood unit. Step 103 provides the erythrocyte deformability data to medical personnel. Step 104 correlates the erythrocyte deformability data with a clinical outcome of a patient that has used the RBC or whole blood unit. Finally, step 105 incorporates the erythrocyte deformability data into subsequent assessments of quality degradation of RBC and whole blood units.

Ektacytometry is a long-established technique for measuring the ability of an individual cell to deform without lysing (breaking), under relatively mild stress—compared to the stress involved in fragility testing. Cell rigidity or loss of plasticity is a key reflection of overall membrane functionality, which is crucial to transfusion success. Once clinically proven to be usefully correlative with post-transfusion cell survival or behavior (with such correlations getting established through the above series of steps), deformability data can then be incorporated into established inventory management techniques such as the Shortest Remaining Shelf Life (SRSL) approach—which long ago replaced FIFO in certain perishable-food applications as the preferred measure of quality.

The main initial users of the method are expected to be technicians employed in hospital blood banks, who could incorporate it into the range of tests routinely performed on blood product. Blood bankers and clinicians would decide how best to utilize the output data for inventory management optimization, product triage, evaluating blood storage or handling methods or conditions, improved protocols, and the like.

Published data indicate that beyond the approximately $800 which hospitals presently spend to acquire and transfuse each RBC unit, they spend much more than this on transfusion-related complications—an often-overlooked cost of transfusion. Hence, any improvement in transfusion efficacy would net significant savings, especially considering the relatively modest cost to be imposed by the testing/analysis.

The anticipated, iterative sequence of implementation for this testing system is to first clinically validate by simultaneously taking measurements in the manner described above while tracking one or more metric of clinical outcomes of transfused patients (e.g. post-transfusion RBC survival, tissue oxygenation, etc.), as well as which RBC unit(s) each patient receives. Based on the clinical outcomes and the respective characterizations of all corresponding samples, candidate models are constructed for correlating the latter to the former. Ultimately, a method of processing all available characteristic data for each sample is chosen to generate some predefined value or set of values for each sample representing its respective unit's prospective transfusion performance level. Note that this value only represents a relative quantitative characterization of the RBC; clinicians must then translate such relative values into determinations of which units are acceptable or preferable for selected patients (or for anyone) under various circumstances. (Similarly for supply or inventory management, blood bankers would use professional judgment in their decisions based upon the test output data.) Recursively, such professional judgments should only improve with time as the testing becomes more established and routine.

The advantages shown in the present disclosure include (without limitation) allowing clinicians to ascertain the severity of degradation of any stored RBC unit near its point of use, and informing the medical judgments of clinicians and blood bankers. Clinicians would know more (besides merely the age) about the degree of efficacy of each RBC unit before deciding whether to use it for a particular patient. For those patients requiring especially high efficacy, this means that some hemodynamically stable patients may receive blood transfusions while at present, under a restrictive protocol, they may not get one—thus facilitating recovery and shortening hospital stays. In other cases, less blood would be required, as in the practice of transfusing additional units in an attempt to compensate for inadequate or unknown unit viability or for an intolerable risk of inadequate performance (liberal protocol). Hence, the risks of complications associated with excessive transfusion will likewise be attenuated. Costs of the additional units, their transfusion, and medical care for the above-noted complications will also be reduced. Furthermore, there is (controversial) research to suggest that for some patients, transfusions of insufficient efficacy can directly cause harm.

As noted, this metric will be useful not only near the point-of-use, but also in hospital inventories and throughout the supply chain and distribution channels to optimize inventory planning and control. Instead of the first-in-first-out (FIFO) system, units can be strategically routed based on real-time data on their respective anticipated viability and measured degradation rates (utilizing existing supply-chain and inventory management strategies and practices); this will improve the aggregate utilization of a scarce and uncertain blood supply. Using ektacytometry brings a particular advantage of having a test that is already being developed in a convenient, clinically-adoptable form for other (unrelated) applications—and thus is readily deployable.

Two clear hurdles remain before general acceptance of this testing would be attained. First, extensive in vitro trials must be performed to confirm the time-independent aspect of RBC degradation, as reflected by membrane deformability. Secondly, in vivo clinical trials will be needed to link measured RBC deformability with relevant clinical outcomes. The link could be challenging to definitively establish, as there is not yet an accepted “gold standard” for determining transfusion “success.” Selection of the proper performance metric(s) and appropriate patient groups will require continued collaboration and consultation with multiple blood bankers and clinicians.

Despite the complexity and scope of the undertaking, there is indeed precedent for such studies ultimately achieving broad acceptance (e.g. TRICC Trial). The expected regulatory hurdles are minor, as no patient contact or product alterations are involved, and no patient would receive anything not already approved under current protocols. In the future, it is conceivable that the 42-day maximum shelf life could be replaced altogether by real-time quality tracking, but initially the testing will simply improve utilization of blood presently being allocated on a FIFO or random basis.

Blood products are valuable and scarce resources, and all efforts must be made to maximize their availability and efficacy. While the need to store RBC remains unavoidable at this time, steps can and should be taken to minimize net quality loss and increase the utilization of all blood supply. An RBC deformability test coupled with appropriate processing and analyses presents such an opportunity (upon validation).

For the purposes of this disclosure, triage is defined as prioritizing the allocation or use of medical resources based on relative patient need.

For the purposes of this disclosure, erythrocyte, also known as a red blood cell, is a blood cell containing hemoglobin and responsible for oxygen delivery to body tissues. The capitalized term Red Blood Cell, or RBC, is the proper name for erythrocytes in storage solution used in transfusion medicine. Also for purposes of this disclosure, RBC includes any stored blood product approved for transfusion which contains erythrocytes.

For the purposes of this disclosure, ektacytometry is the use of laser-assisted diffraction to measure the deformability of a cell.

For the purposes of this disclosure, an ektacytometer is any device which performs ektacytometery, as defined above.

For the purposes of this disclosure, cell deformability is its ability to be manipulated or re-shaped without rupturing or breaking the membrane (lysis, or hemolysis for erythrocytes); by contrast, cell membrane fragility is a propensity for lysis, and thus typically involves exposure to higher stresses in order to ascertain.

While these descriptions of the invention enable one of ordinary skill to use what are considered presently to be the best known modes of every respective aspect thereof, those in the field will also understand and appreciate the existence of variations, combinations, and equivalents of the specific embodiments, methods, and/or examples used. Many variations in form and uses would occur to those skilled in the art. Potential variations include using various kinds of ektacytometers (and related devices and/or techniques), various existing statistical and computational processes, various known inventory management strategies, and various triaging approaches. All such and other variations are intended to be within the scope and spirit of the invention.

We claim:

1. A method for assessing the quality degradation of an RBC or whole blood unit, the method comprising:
using an ektacytometer to generate erythrocyte deformability data for the RBC or whole blood unit;
analyzing the erythrocyte deformability data to assess the quality degradation of the RBC or whole blood unit;
providing the erythrocyte deformability data to medical personnel;
correlating the erythrocyte deformability data with a clinical outcome of a patient that has used the RBC or whole blood unit; and
incorporating the erythrocyte deformability data into subsequent assessments of quality degradation of RBC and whole blood units.

2. The method of claim 1, wherein the deformability data comprises membrane deformability as a function of time.

3. The method of claim 1, wherein the deformability data comprises the rate of change of membrane deformability as a function of time.

4. The method of claim 1, further comprising using deformability data to generate a value representing RBC or whole blood unit suitability for transfusion or degree of prospective efficacy.

5. The method of claim 1 further comprising assessing the quality degradation of an RBC or whole blood unit in some combination with RBC fragility, morphologic RBC parameters, or metabolic RBC parameters to evaluate the RBC or whole blood unit.

6. The method of claim 1 further comprising using the quality degradation of an RBC or whole blood unit to aid management of blood supply or inventory.

7. The method of claim 1 further comprising using the quality degradation of an RBC or whole blood unit to evaluate blood storage and handling methods.

8. The method of claim 1 further comprising using the quality degradation of an RBC or whole blood unit to aid RBC or whole blood usage in triage.

9. The method of claim 1 further comprising using the quality degradation of an RBC or whole blood unit to make indication-specific assessments of RBC unit suitability for specific patient groups, based on particularized efficacy correlations found for such patient groups.
